(19) United States
(12) Patent Application Publication
(10) Pub. No.: US 2022/0380439 A1
(43) Pub. Date: Dec. 1, 2022

(54) PURIFICATION OF FVIII FROM PLASMA USING SILICON OXIDE ADSORPTION

(71) Applicant: Takeda Pharmaceutical Company Limited, Osaka (JP)

(72) Inventors: Hany Anwar Hassouna, Moorpark, CA (US); Joshua Elliot Yu, San Francisco, CA (US); Alexander Zaydenberg, Chatsworth, CA (US); Ani Der-Sarkissian, Northridge, CA (US); Yasser Badour, Rolling Hills Estates, CA (US); Emily Vincent, San Jose, CA (US); Patrick D. Gavit, Glendora, CA (US)

(21) Appl. No.: 17/556,857

(22) Filed: Dec. 20, 2021

Related U.S. Application Data
(60) Provisional application No. 63/111,191, filed on Nov. 9, 2020.

(51) Int. Cl. C07K 14/75; B01D 35/00; A61M 1/02

(52) U.S. Cl. CPC C07K 14/75; B01D 35/00; A61M 1/0218

(57) ABSTRACT

Efficient methods for capture and removal of fibrinogen from blood plasma fractions, especially cryoprecipitate, and Fraction II+III providing high yields of blood coagulation Factor VIII are disclosed. According to this disclosure, there is provided a method of separating plasma cryoprecipitate or Fraction II+III comprising a blood coagulation factor and fibrinogen into a first fraction comprising the blood coagulation factor and a second fraction containing the fibrinogen, the method comprising: (a) contacting he plasma cryoprecipitate with solid SiO₂ or Al(OH)₃, thereby adsorbing the fibrinogen onto the solid SiO₂ or Al(OH)₃; and (b) separating the fibrinogen adsorbed onto the solid SiO₂ or Al(OH)₃ from the blood factor, thereby forming the first fraction and the second fraction.

### CROSS REFERENCES TO APPLICATIONS

This application claims priority to U.S. Provisional Patent Application No. 63/111,191 filed Nov. 9, 2020, the disclosure of which is hereby incorporated herein by reference in its entirety for all purposes.

### BACKGROUND OF THE INVENTION

Blood, blood plasma, and blood serum are extremely complicated protein-containing solutions that comprise many types of compounds other than protein(s), all carefully balanced and regulated to work in a very broad range of biochemically complicated functions such as oxygen ⁻transport, immune response, and coagulation, When blood is drawn from a subject and exposed to the atmosphere it becomes highly unstable, and methods of purifying or enriching a blood protein must take account of this fact. Although chemical agents, such as heparin and sodium citrate, are added to increase the stability and, to a certain degree, prevent coagulation of the plasma obtained by separating blood, plasma is a very fragile, highly concentrated and viscous protein solution also comprising significant amounts of lipids. Despite the addition of stabilizers any handling or alteration of the plasma composition involves the risk of accidental destabilization, which may cause activation of the coagulation cascade, precipitation of, e.g,, lipid components as well as denaturation of protein(s), thereby making the blood very difficult to work with. Any method employed to isolate proteins from blood or blood derived solutions must take the inherent instability of the solution and the proteins themselves into consideration. This has proven to be a very significant challenge for the large-scale production of therapeutic products from blood.

The complexity and instability of the blood makes the separation and isolation of blood proteins much more complicated and economically demanding than the isolation of proteins from other protein solution, such as cell culture supernatants and fermentation broths typically used in the biotech industry. Also, the biotech industry will typically isolate only one specific product from a cell culture supernatant, while for economical and ethical reasons the therapeutic blood fractionation industry generally must isolate as many products as possible from the limited amount of blood available.

The cost of blood, serum and plasma has increased significantly, one reason being increased cost of the safety measures needed to prevent infectious disease, e.g., viral diseases spreading from blood donors to recipients of the blood products. The high cost of plasma as well as increased costs of viral elimination steps and other safety measures during processing has put the blood fractionation industry under a significant pressure to increase the yield of individual products such as coagulation factors, e.g. Factor VIII. The long felt needs of the blood fractionation industry are difficult to satisfy with known technology and, although attempts have been made to employ modern adsorption techniques as an alternative to the established precipitation methods there are still significant problems in terms of economical feasibility and processing robustness of hitherto described adsorption methods. Human and animal blood comprises many proteins and enzymes, which possess therapeutic and potentially lifesaving properties. Some of these proteins may be found in the red blood cells whereas others are found in solution in plasma or serum. Since the middle of the 20th century such proteins have been the target for large-scale and specific isolation with the aim of purifying and standardizing the proteins for use as human therapeutic agents. Some proteins from plasma are produced in the scale of several thousand kg per year (albumin and IgG) while others are produced only in the gram to kilogram per year scale. However, on a worldwide basis many million liters of blood per year are processed to isolate these proteins.

One of the conventionally used methods for the fractionation of blood plasma or blood serum protein(s) is described in U.S. Pat. No. 2,390,074 (Cohn et al.) which discloses a method for the fractionation of plasma or serum proteins in large-scale which utilize ethanol precipitation and regulates temperature, pH, ionic strength and time to control precipitation of certain proteins from human plasma. The fractionation method involves the stepwise addition of ethanol to the plasma raw material in order to obtain several precipitates (fractions) and corresponding supernatants comprising different enriched protein solutions. A drawback of the ethanol precipitation method disclosed by Cohn et al. is that some proteins tend to denature during the process resulting in decreased yield of protein and contamination with aggregates that must be removed before an acceptable therapeutic product can be obtained. Furthermore, during the Cohn fractionation process, precipitated proteins must be resolubili zed for further processing. Such resolubilized protein solutions may comprise significant levels of insoluble (denatured) protein and lipid material that makes it difficult and time consuming to prepare the target product, which also contributes significantly to the loss of valuable product. Additionally, in this process a specific protein may distribute into several of the fractions obtained during the stepwise addition of ethanol, again resulting in low yields and time-consuming preparation of re-combined protein fractions.

Factor VIII (FVIII) is a protein found in blood plasma, which acts as a cofactor in the cascade of reactions leading to blood coagulation. A deficiency in FVIII activity results in the clotting disorder known as hemophilia A, an inherited condition primarily affecting males. Hemophilia A is currently treated with therapeutic preparations of FVIII derived from human plasma or manufactured using recombinant DNA technology. Such preparations are administered either in response to a bleeding episode (on-demand therapy) or at frequent, regular intervals to prevent uncontrolled bleeding (prophylaxis).

Several different methods are known for the production of antihemophilic factor (AHF or Factor VIII) for therapeutic use, e.g.. selective precipitation, batch absorption and elution, extraction in low ionic media and chromatography. While purification of Factor VIII eliminates a variety of other plasma proteins, fibrinogen is by far the most important and troublesome of these proteins, particularly when denatured during its processing. Denatured fibrinogen impairs filtration of FVIII, causes appreciable losses of FVIII during purification steps and decreases the solubility of the lyophilized product in reconstituting fluid. A satisfactory method of purifying FVIII requires removal of appreciable quantities of fibrinogen. The selective precipitation techniques described above accomplish this purpose but have the disadvantages of either further denaturing fibrinogen and FVIII or producing undesirable losses of FVIII. See, U. S. Pat. No. 4,789,733.

Procedures involving extracting Factor VIII from cryoprecipitate in low ionic strength buffers, while decreasing fibrinogen content of the final product somewhat, still lead to an undesirably high protein content in the product, require special equipment and procedures for centrifugation and are limited in the total amount of Mill extracted from cryoprecipitate without impairing purification.

Other problems commonly associated with large-scale manufacture of FVIII are contamination of the final product by pyrogenic substances and viruses which cause hepatitis. With chemical precipitants these undesirable contaminants may actually be enhanced.

Presently used isolation and purification processes could be improved because trace impurities resulting from inefficient purification processes may be able to stimulate an immune response in patients. Furthermore, purification processes that fail to separate active and inactive part of the product, as the presently used processes, can lead to a product with unpredictable efficacy and a specific activity, which varies between separate lots.

U.S. Pat. No. 4,387,092 describes a method of purifying FVIII from fibrinogen by a method including precipitation of fibrinogen with 6% polyol at 0-5° C. In the method described in U.S. Pat. No. 4,188,318, FVII1 is purified by a specific method for the large-scale manufacture of a Factor VIII concentrate using selective cold precipitation of excessive amounts of fibrinogen, its denatured forms and degradation products in low ionic strength solution, without added chemicals, and without undesirable loss of AHF activity. U.S. Pat. No. 8,563,288 describes removal of plasminogen from a crude blood coagulation preparation by treatment with a rigid amino acid immobilized on a chromatographic resin. U.S. Patent Application Publication No. 2015/0158906 discloses a method passing a feedstock comprising fibrinogen and/or Factor VIII and /or VWF through a hydrophobic charge-induction chromatographic (HCIC) resin and recovering the solution comprising fibrinogen and/or Factor VIII and/or VWF that passes through the resin to reduce the destabilizing level of plasminogen and/or tissue plasminogen activator and/or other protease(s) in the solution.

U.S. Patent Application Publication No. 2017/0282095 describes a method of isolating proteins from blood supplemented with alcohol using expanded bed chromatography on a solid support functionalized with a ligand.

There are also other methods of producing FVIII-concentrates, i.e. the treatment of the plasma with adsorption agents, such asflorigel, bentonite, ion exchangers, and permeation-chromatographic methods. Such products are referred to as “highly purified AHF” or “High Purity AHF.” Their factor-VIII-activity, however, also is only 90 to 100 times higher than that of native plasma, while the content of factor-Mill-inactive proteins (fibrinogen) still amounts to 35% of the total protein. See, e.g., U.S. Pat. No. 4,404,131.

An exemplary known process is set forth in FIG. 2. Plasma cryoprecipitate paste is suspended in water for injection in a ratio of 1 (kg): 3.5(L) cryoprecipitate paste: water, forming Sample A. Sample A is made isoelectric to FVIII and a cold precipitation step is performed to precipitate some of the fibrinogen and other proteins. This step includes adding CaCl₂ at 0.04 M, and adjusting the pH to 6.5-7.11, and adjusting the temperature to 8-12° C., and the resulting suspension is clarified by centrifugation. The bulk supernatant is drawn off and stabilized by raising the pH to 7.2-7.6, and adjusting the temperature to 18-26° C., forming Sample B. Sample B is submitted to NaCl addition, adjusting the NaCl content to 0.8 M, and CaCl₂ is added to a concentration of 0.05 M. The resulting solution is clarified by filtration, resulting in Clarified Bulk, This solution is submitted to a viral reduction step using solvent/detergent treatment. A solvent (tri-(n-butyl) phosphate) and detergent (Octoxynol 9) is added to Clarified Bulk to a final concentration of 1.0% (v/v) and 0.3% (v/v), respectively, forming Sample C. Sample C is loaded onto an immunoaffinity column specifically binding FVIII.

Though, attempts to develop new industrial FVIII purification processes have provided incremental advances and attained some increased yield of FVIII, these processes are complex, touchy procedures, lacking robustness and having other difficulties related to the cold drop that are a challenge to balancing the yield and economy of these processes. Elimination of the cold drop would be a significant advance in the art as this step generates a sticky precipitate that can clog lines, is difficult to remove and makes recovery of Factor VIII complex.

As set forth herein, the present invention solves a number of shortcomings of prior methods.

### BRIEF SUMMARY OF THE INVENTION

The invention avoids the disadvantages and difficulties pointed out above and provides a FVIII formulation in which the non-FVIII -active proteins, particularly fibrinogen, are essentially completely removed from the FVIII. By eliminating, inter alia, the cold drop, the present method further overcomes current process and equipment challenges associated with precipitated fibrinogen, and provides a method of removing fibrinogen upstream from the immunoaffinity column.

The FVIII-activity of the formulation of the invention is high, based on the protein present in the concentrate. By eliminating cooling and pH adjustment steps long thought to be critical to the successful isolation of FVIII, the present invention quite surprisingly provides a quicker, simpler, more economical process for recovery of FVIII than previous methods. The method of the invention eliminates cold precipitation and centrifugation steps; the cold precipitation step is exacting and time-consuming, and centrifuges are a capital-intensive investment and expensive to maintain and repair. The simpler process of the invention leads to a. reduction of process cycle steps and cycle time, which is a significant improvement over prior methods.

In an exemplary embodiment, the invention solves the problem of removing ⁻fibrinogen from FVIII without the use of a centrifuge. In various embodiments, the invention solves the problem of removing fibrinogen from FVIII in a process without a cold precipitation step. In some embodiments, the invention solves the problem of separating fibrinogen from FVIII without a laborious process of cooling the mixture of FVIII and fibrinogen while adjusting downwards the pH of mixture. An exemplary process of the invention solves the problem of collecting fibrinogen after its separation from FVIII using a method described herein.

Previous methods of enriching FVIII from a plasma-derived FVIII-enriched starting material involve slowly altering the pH of the starting material (e.g., cryosuspension) to 6.7 with 1M acetic acid and gradually cooling the suspension to 9.5° C. as a precipitation occurs over a period of continuous mixing. Cooling and reducing the pH requires about 2.5 hours, and carries the risk of Factor VIII precipitation if performed incorrectly. During this process, a predominately fibrinogen and fibronectin precipitate is formed, accounting for greater than about 50% of the total protein. The precipitate is removed by centrifugation at 3000×g for about 120 minutes at 9.5° C. The pH of the resulting cold supernatant is then adjusted upwards to 7.4 with 1N NaOH in a process performed over about 1.5 hour. Surprisingly, the process disclosed herein eliminates each of these exacting and time-consuming steps to provide a simple, repeatable process that is more economical than prior art processes.

In an exemplary embodiment, the method of the invention eliminates the cold precipitation step from a method of separating fibrinogen from FVIII The cold precipitation step is replaced by contacting an a.queous suspension of cryoprecipitate or Fraction II+III with finely divided silica (SiO₂) or alumina (Al(OH₃)), and filtering the resulting suspension. Fibrinogen is adsorbed onto the silica or alumina and easily separated from FVIII remaining in solution.

Thus, in an exemplary embodiment, there is provided a method of separating plasma cryoprecipitate comprising a blood coagulation factor and fibrinogen into a first fraction comprising the blood coagulation factor and a second fraction containing the fibrinogen, the method comprising: (a) contacting the plasma cryoprecipitate with solid SiO₂ or Al(OH)₃, thereby adsorbing the fibrinogen onto the solid SiO₂; and (b) separating the fibrinogen adsorbed onto the solid SiO₂ or Al(OH)₃ from the blood factor, thereby forming the first fraction and the second fraction.

In an exemplary embodiment, there is provided a method of separating Cohn Fraction II+III, comprising a blood coagulation factor and fibrinogen into a first fraction comprising the blood coagulation factor and a second fraction containing the fibrinogen, the method comprising: (a) contacting the plasma cryoprecipitate with solid SiO₂ or Al(OH)₃, thereby adsorbing the fibrinogen onto the solid SiO₂; and (b) separating the fibrinogen adsorbed onto the solid SiO₂ or Al(OH)₃ from the blood factor, thereby forming the first fraction and the second fraction.

An exemplary object of the invention to provide a method of producing a FVIII (AHF)-high-concentrate with a specific activity of at least 2.5 units AHF and a fibrinogen content of less than 0.25 mg/mg protein, which method is applicable on an industrial and technical scale, ensuring a high economy.

In various embodiments, the invention provides a composition containing FVIII enriched by a process of the invention, a pharmaceutical formulation containing this composition, including a unit dosage formulation, and a method of treating or preventing a disease in subject in need thereof by administering to the subject a pharmaceutical formulation of the invention.

### DESCRIPTION OF THE FIGURES

FIG. 1 is a process schematic for an exemplary embodiment of the invention.

FIG. 2 is a flow chart comparing an exemplary method of the invention with that of a production method for a marketed FVIII formulation.

### DETAILED DESCRIPTION OF THE INVENTION

### I. Introduction

Given the broad use of therapeutic plasma-derived blood protein compositions, such as blood coagulation factors, coagulation factor inhibitors, immune globulin compositions, and proteins of the complement system, ensuring the efficacy and safety of these compositions is of paramount importance.

Furthermore, unlike other biologics produced via recombinant expression of DNA vectors in host cell lines, plasma-derived proteins are fractionated from human blood and plasma donations. Thus, the supply of these products cannot be increased by simply increasing the volume of production. Rather the level of commercially available blood products is limited by the available supply of blood and plasma donations. This dynamic results in a crimp in the supply chain at the availability of raw human plasma for the manufacture of plasma-derived blood factors.

In certain aspects, the present invention provides manufacturing methods based on the surprising finding that finely divided silicon dioxide (SiO₂) can be used to remove significant amounts of fibrinogen from plasma-derived protein solutions. An exemplary solution contains fibrinogen and one or more blood factors, e.g., FVIII. The product resulting from a method of the invention is a FVIII preparation having less fibrinogen in it than was present in the starting plasma-derived solution.

The methods provided herein are easily integrated into existing manufacturing procedures, for example, the fractionation of pooled plasma samples, preferably human plasma samples, by ethanol in the cold (reviewed in Schultze H E, Heremans J F; MOLECULAR BIOLOGY OF HUMAN PROTEINS. VOLUME I: NATURE AND METABOLISM OF EXTRACELLULAR PROTEINS 1966, Elsevier Publishing Company; p. 236-317). However, the methods provided herein are in no way limited in their use to manufacturing methods including ethanol fractionation. Other methodologies for the purification of plasma-derived proteins are also compatible with the methods provided herein, for example, polymer (e.g., PEG) fractionation and chromatographic methodologies (e.g., anion and/or cation exchange chromatography, affinity chromatography, immuno-affinity chromatography, size exclusion chromatography, hydrophobic interaction chromatography, mixed mode chromatography, and the like).

### II. Definitions

Unless defined otherwise, all technical and scientific terms used herein generally have the same meaning as commonly understood by one of ordinary skill in the art to which this invention belongs. Generally, the nomenclature used herein and the laboratory procedures in organic chemistry, pharmaceutical formulation, and medical imaging are those well-known and commonly employed in the art.

The articles “a” and “an” are used herein to refer to one or to more than one (i.e. to at least one) of the grammatical object of the article.

The methods and techniques of the present invention are generally performed according to conventional methods well known in the art and as described in various general and more specific references that are cited and discussed throughout the present specification unless otherwise indicated. See, e.g., Sambrook et al. MOLECULAR CLONING: A LABORATORY MANUAL, SECOND ED., Cold Spring Harbor Laboratory Press, Cold Spring Harbor, N.Y. (1989) and Ausubel et al., CURRENT PROTOCOLS IN MOLECULAR BIOLOGY, GREENE PUBLISHING ASSOCIATES (1992), and Harlow and Lane, ANTIBODIES: A LABORATORY MANUAL, Cold Spring Harbor Laboratory Press, Cold Spring Harbor, N,Y. (1990), incorporated herein by reference, Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The following terms, unless otherwise indicated, are understood to have the following meanings.

Herein, the term “Factor VIII” or “FVIII” or “FVIII” refers to any FVIII molecule which has at least a portion of the B domain intact, and which exhibits biological activity that is associated with native FVIII. In one embodiment of the disclosure, the FVIII molecule is full-length FVIII. In general, FVIII, as used herein, refers to naturally occurring FVIII obtained from plasma. In an exemplary embodiment, the FVIII is in a formulation essentially free of plasminogen and has been so rendered by a method of the invention.

As used herein, “plasma-derived FVIII” or “plasmatic” includes all forms of the protein found in blood obtained from a mammal having the property of activating the coagulation pathway.

The term “isolated protein”, or “isolated polypeptide” is a protein that by virtue of its origin or source of derivation (1) is essentially free from naturally associated components that accompany it in its native state, or (2) is essentially free of other proteins from the same species. Thus, a polypeptide that is chemically synthesized or synthesized in a cellular system different from the cell from which it naturally originates will be “isolated” from its naturally associated components. A protein may also be rendered essentially free of naturally-associated components by isolation or enrichment, using protein purification techniques well known in the art or those disclosed herein.

In an exemplary embodiment, the invention provides an isolated FVIII. In various embodiments, the invention provides an isolated FVIII preparation, which is a product of a method of the invention. An exemplary preparation has less fibrinogen present than was present in the starting composition entering the method of the invention (e.g., plasma, Cryoprecipitate). In some embodiments, the FVIII preparation emerging from the method of the invention is substantially free of fibrinogen. In some embodiments, the FYIII preparation downstream from the silica treatment and upstream from the immunoaffinity column is substantially free of fibrinogen.

As used herein, “substantially free of fibrinogen” refers to a preparation containing FVIII with less than about 1%. less than about 5%, or less than about 10% of the fibrinogen present in the starting plasma. In an exemplary embodiment, the preparation containing Fan includes less than about 1%, less than about 5%, or less than about 10% of the fibrinogen present in the Cryoprecipitate starting material.

In an exemplary embodiment, the preparation is the product of treating a process intermediate with silicon dioxide as discussed herein. An exemplary preparation contains FVIII and less than about 1%, less than about 5%, or less than about 10% of the fibrinogen present in the plasma or Cryoprecipitate starting material.

An exemplary “isolated” human FVIII is a human FVIII that is at least about 90% pure (i.e., does not contain more than 10% protein impurity). Preferably, an isolated human FVIII is at least about 95%, 98%, 99% or at least about 99.5% pure. An exemplary isolated human FVIII is prepared by a method of the invention as set forth herein. An exemplary process of the invention provides FVIII about as pure and about as active as currently accepted industrial manufacturing processes for preparing this protein for human administration. In an exemplary process the FVIII activity in the final container ranges between about 20 and about 200

In various embodiments, the FVIII emerging from a solvent/detergent treatment step of an exemplary process of the invention is an “isolated human FVIII”. In an exemplary embodiment, the emerging from an affinity chromatography step downstream of the solvent/detergent treatment step of an exemplary process of the invention is an “isolated human FVIII”.

As used herein, the term “substantial fraction” refers to at least 10% of the population of a particular protein in a composition. For example, when referring to a substantial fraction of fibtinogen being removed from a composition, a substantial fraction of fibtinogen corresponds to at least about 10%, e.g., at least about 50, 55, 60, 65, 70, 75, 80, 85, 90, 95, 96, 97, 98, at least about 99%, or more of the fibinrogen present in a starting composition (e.g., plasma, Cryoprecipitate) being removed by a process of the invention (or a component step of the invention, e.g., treatment with silicon dioxide) and, therefore, absent in the product of the invention.

As used herein, a “Cohn pool” refers to the starting material used for the fractionation of a plasma sample or pool of plasma samples. Cohn pools include whole plasma, cryo-poor plasma samples, and pools of cryo-poor plasma samples that may or may not have been subjected to a pre-processing step, a fractionation step or a combination thereof. In certain embodiments, a Cohn pool is a cryo-poor plasma sample from which one or more blood factor have been removed in a pre-processing step, for example, adsorption onto a solid phase (e.g., aluminum hydroxide, finely divided silicon dioxide, etc.), or chromatographic step (e.g., ion exchange or heparin affinity chromatography). Various blood factors, including but not limited to Factor Eight Inhibitor Bypass Activity (FEIBA), Factor IX-complex, Factor VII-concentrate, or Antithrombin 11I-complex, may be isolated from the cryo-poor plasma sample to form a Cohn pool.

The term “cryoprecipitate”, also referred to as cryoprecipitated anti-haemophilic factor (AHF), refers to a blood product produced by controlled thawing of frozen plasma (e.g., whole blood-derived fresh frozen plasma, apheresis derived plasma) to form a precipitate comprising one or more coagulation factors including, without limitation, fibrinogen, Factor VIII. Factor XIII, vWF, and/or fibronectin. Such cryoprecipitate is prepared by slow, controlled thawing of frozen plasma (e.g., whole blood-derived fresh frozen plasma, or FFP), for example between 1° and 6° C. (e.g., 4±2° C.), which results in the formation of a white precipitate, and then recovering the precipitate following separation from the liquid pla.stna portion, also referred to herein as “supernatant,” such as by refrigerated centrifugation. The “cryo-poor” remaining plasma, also referred to herein as “cryo-poor plasma” (CPP), “cryoprecipitate-reduced plasma,” or “ctyosupernatant,” is removed from the bag, and the isolated cold-insoluble precipitate is re-suspended in a portion of the plasma left behind and generally re-frozen within 1 hour, and. stored frozen until needed for transtlision. A cryoprecipitate may he resuspended in any suitable volume of plasma after recovery. Cryoprecipitate (also known as “cryo”) is a blood product comprising a portion of plasma rich in coagulation factors.

Cryoprecipitate serves as a source of fibrinogen, Factor VIII, Factor XIII, vWF, and fibronectin. This component is used in the control of bleeding associated with fibrinogen deficiency and to treat Factor XIII deficiency when volume considerations preclude the use of frozen plasma and recombinant proteins are not available. It is also indicated as second-line therapy for von Willebrand disease and hemophilia A (Factor VIII deficiency). Coagulation factor preparations other than cryoprecipitate are generally preferred when blood component therapy is needed for management of von Willebrand disease and Factor VIII deficiency. Although many uses of cryoprecipitate products have been replaced by factor concentrates or recombinant factors, cryo is still routinely stocked by many hospital blood banks for use in the replacement of fibrinogen in patients, such as, for example, patients with acquired hypofibrinogenemia and bleeding (e.g., massive hemorrhage). Compatibility testing of blood groups is not strictly necessary for cryoprecipitate; however, transfusion of ABO-compatible cryo is generally preferred when possible. Methods for preparing a cryoprecipitate are well known in the art.

As used herein, a “cryoprecipitate filter cake” refers to a solid recovered after filtration of an aqueous suspension of a cryoprecipitate paste including finely divided SiO₂ (or Al(OH₃)). A cryoprecipitate suspension is treated with an adsorptive material, for example, finely divided silica, to remove impurities such as fibrinogen. In another preferred embodiment, filter aid is added to the cryoprecipitate suspension prior to filtration, In an exemplary embodiment, a, cryoprecipitate suspension is treated with both an adsorptive material and a filter aid prior to centrifugation or filtration. Upon separation of the cryoprecipitate suspension supernatant, the recovered solid material is referred to as the “cryoprecipitate filter cake”.

As used herein, a “Fraction II+III filter cake” refers to a solid recovered after the filtration or centrifugation of a Cohn-Oncley or equivalent Fraction paste suspension. A Fraction suspension is treated with an adsorptive material, for example, finely divided silica, to remove impurities such as fibrinogen. In another preferred embodiment, filter aid is added to the Fraction II+III suspension prior to filtration. In an exemplary embodiment, a Fraction II+III suspension is treated with both an adsorptive material and a filter aid prior to centrifugation or filtration. Upon separation of the clarified Fraction II+III suspension supernatant, the recovered solid phase material is referred to as the “Fraction II+III filter cake”.

As used herein, “cryo-poor plasma” refers to the supernatant created after the removal of cryo-precipitate formed by thawing plasma or pooled plasma at temperatures near freezing, e.g., at temperatures below about 10° C., preferably at a temperature no higher than about 6° C. Cryo-precipitation is commonly performed, for example, by thawing previously frozen pooled plasma, which has already been assayed for safety and quality considerations, although fresh plasma may also be used. After complete thawing of the frozen plasma at low temperature, separation of the solid cryo-precipitates from the liquid supernatant is performed in the cold (e.g., ≤6° C.) by centrifugation of filtration.

The term “plasma” refers to any plasma blood product known in the art. In the context of the present invention, plasma may refer interchangeably to recovered plasma (i.e., plasma that has been separated from whole blood ex vivo) or source plasma (i.e., plasma collected via plasmapheresis). In some embodiments, plasma refers to whole blood-derived fresh frozen plasma. In some embodiments, plasma refers to one or more plasma units from a whole blood donation (e.g, approximately 180-250 mL volume each). In some embodiments, plasma refers to one or more plasma units from an apheresis blood donation (may be up to approximately 700-800 mL each). In some embodiments, plasma refers to a single unit. In some embodiments, plasma is pooled from multiple units. In some embodiments, plasma may contain one or more additional components, including, without limitation, one or more pathogen-inactivation compounds and/or byproducts of a pathogen-inactivation process.

In the present context the term “supernatant” relates to a liquid fraction, which lies above a sediment fraction or a precipitated fraction. The liquid fraction may be either a combination of compounds or a pure compound. In an embodiment of the present invention a fraction used or produced by the method may be in the form of a liquid (a liquid fraction), a sediment (a sediment fraction) or a precipitate (a precipitated fraction).

As used herein, “silicon dioxide” or “finely divided silica” refers to an oxide of silicon having the formula Sift, manufactured in a fashion that allows for the adsorption of fibrinogen onto its surface. Exemplary forms of silicon dioxide suitable for use in the methods of the present invention include, without limitation, fumed silica, pyrogenic silica, Aerosiltmt, Cab-O-Sil, colloidal silica, diatomaceous earth, and the like. In a preferred embodiment, a commercial hydrophilic fumed silica product is used for the methods provided herein. Non-limiting examples of these products include those marketed by Evonik Industries under the trade name Aerosil® (e.g., Aerosil 90, Aerosi1 130, Aerosil 150, Aerosil 200, Aerosil 300, Aerosi1 380, Aerosil OX 50, Aerosil EG 50, Aerosil TT 600, Aerosil 200 SP, Aerosil 300 SP, Aerosil 300/30, and Aerosil® 380). In an exemplary embodiment, the SiO₂ is Aerosil® 380. Though different compounds, the method of the invention can also be practiced with Al(OH)₃ instead of or in addition to silicon dioxide. When one of these species is referred to, this reference is understood to refer to either of these filter aids and a combination thereof.

The term, “filter aid”, as used herein, refers to additives which are used in solid-liquid separation processes in order to facilitate deposition of the solids with simultaneously sufficient permeability of the resultant filter cake by formation of a porous precoat layer on the actual filter medium and/or by incorporation into the filter cake structure. Exemplary filter aids include kieselguhr, perlite, aluminum oxide, glass, plant granules, wood fibers and/or cellulose or mixtures thereof.

Kieselguhr is a pulverulent substance principally comprising the silicone dioxide shells of fossil diatoms which have a very porous structure. Commercially, kieselguhr can be obtained, for example, from the companies Lehmann and Voss (for example Celite®), Dicelite or PallSeitzSchenk. Perlite filter aids comprise volcanic obsidian rock and are produced by thermal expansion; chemically these are aluminum silicate, which is almost as inert as silica. The structure of perlite filter aids corresponds to spherical fragments not having the same porosity, as is the case with the filigree skeleton of diatoms. Commercially, perlite can be obtained, for example, from the companies Lehmann and Voss (Harbolite®) and Dicelite.

Preconditioned natural fibers from extract-free cellulose which are in part specially prepared in order to ensure high purifies and also odor and flavor neutrality can likewise be used as filter aids. Cellulose filter aids are mechanically and chemically very stable, insoluble in virtually all media and almost pH neutral. Commercially they are distributed, for example, by J. Retternmaier & Sohne (for example Arbocelg, Filtracel® and Vitacel® types).

In an exemplary embodiment, the filter aid is Celpure® C300.

As used herein, the term “filtration” encompasses a variety of sieving and membrane filtration methods in which hydrostatic pressure forces a liquid against a semi-permeable filter. Suspended solids and, while water and solutes pass through the filter. An exemplary filtration technique of use in the invention is tangential filtration. Exemplary filtration in the context of the invention produces a Factor II+II filter cake or a cryoprecipitate filter cake.

As used herein, the term “mixing” describes an act of causing essentially equal distribution of two or more distinct compounds or substances in a solution or suspension by any form of agitation. Complete equal distribution of all ingredients in a solution or suspension may result but is not required as a result of “mixing” as the term is used in this application.

As used herein, the term “solvent” encompasses any liquid substance capable of dissolving or dispersing one or more other substances. A solvent may be inorganic in nature, such as water, or it may be an organic liquid, such as ethanol, acetone, methyl acetate, ethyl acetate, hexane, petrol ether, etc.

As used herein, the term “detergent” is used in this application interchangeably with the term “surfactant” or “surface acting agent,” Surfactants are typically organic compounds that are amphiphilic, i.e., containing both hydrophobic groups (“tails”) and hydrophilic groups (“heads”), which render surfactants soluble in both organic solvents and water. A surfactant can be classified by the presence of formally charged groups in its head. A non-ionic surfactant has no charge groups in its head, whereas an ionic surfactant carries a net charge in its head. A zwitterionic surfactant contains a head with two oppositely charged groups. Some examples of common surfactants include: Anionic (based on sulfate, sulfonate or carboxylate anions): perfiuorooctanoate (PFOA or PFO), perfluorooctanesulfonate (PFOS), sodium dodecyl sulfate (SDS), ammonium lauryl sulfate, and other alkyl sulfate salts, sodium laureth sulfate (also known as sodium lauryl ether sulfate, or SLES), alkyl benzene sulfonate; cationic (based on quaternary ammonium cations): cetyl trimethylammonium bromide (CTAB) a.k.a. hexadecyl trimethyl ammonium bromide, and other alkyltrimethylammonium salts, cetylpyridinium chloride (CPC), polyethoxylated tallow amine (POEA), benzalkonium chloride (BAC), benzethonium chloride (BZT); long chain fatty acids and their salts: including caprylate, caprylic acid, heptanoat, hexanoic acid, heptanoic acid, nanoic acid, decanoic acid, and the like; Zwitterionic (amphoteric): dodecyl betaine; cocamidopropyl betaine; coco ampho glycinate; nonionic: alkyl poly(ethylene oxide), alkylphenol poly(ethylene oxide), copolymers of poly(ethylene oxide) and polypropylene oxide) (commercially known as Poloxamers or Poloxamines), alkyl polyglucosides, including octyl glucoside, decyl maltoside, fatty alcohols (e.g., cetyl alcohol and oleyl alcohol), cocamide MEA, cocamide DEA, polysorbates (Tween 20, Tween 80, etc.), Triton detergents, and dodecyl dimethylamine oxide. An exemplary detergent is Octoxynol 9.

Exemplary embodiments incorporate a “solvent/detergent treatment” of at least one product downstream from the filtration step removing the adsorptive material, e.g., silica. An exemplary “solvent detergent treatment,” utilizes an organic solvent (e.g., tri-N-butyl phosphate), which is part of the solvent detergent mixture used to inactivate lipid-enveloped viruses in solution. An exemplary detergent used in this treatment is Octoxynol 9.

The term “polypeptide” or “protein” encompasses native or artificial proteins, protein fragments and polypeptide analogs of a protein sequence. A polypeptide may be monomeric or polymeric. Exemplary polypeptides or proteins include human and human fibrinogen.

As used herein a “polypeptide” refers to a polymer composed of amino acid residues, structural variants, related naturally-occurring structural variants, and synthetic non-naturally occurring analogs thereof linked via peptide bonds, Synthetic polypeptides are prepared, for example, using an automated polypeptide synthesizer. The term “protein” typically refers to large polypeptides. The term “peptide” typically refers to short polypeptides.

A “naturally-occurring” FVIII polypeptide sequence is typically from a mammal including, but not limited to, primate, e.g., human; rodent, e.g., rat, mouse, hamster; cow, pig, horse, sheep, or any mammal, Reference polynucleotide and polypeptide sequences include, e,g., UniProtKB/Swiss-Prot P00451 (FA8_HUMAN); Gitschier et al., Characterization of the human Factor VIII gene, Nature, 312(5992): 326-30 (1984): Vehar G H et at, Structure of human Factor VIII, Nature, 312(5992):337-42 (1984); and Thompson A R. Structure and Function of the Factor VIII gene and protein, ,Sean Thromb Heniosi, 2003:29; 11-29 (2002), (references incorporated herein in their entireties).

As used herein, an “antibody” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes, Light chains are classified as either kappa or lambda. Heavy chains are classified as gamma, mu, alpha, delta, or epsilon, which in turn define the immunoglobulin classes, IgG, IgM, IgA, IgD, and IgE, respectively. An exemplary immunoglobulin (antibody) structural unit is composed of two pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (V_{H}) refer to these light and heavy chains respectively. An exemplary antibody is a monoclonal antibody specifically binding to FVIII. In various embodiments, the antibody specifically binds to FVIII, e.g., human FVIII in a preparation downstream of the filtration step, e.g., emerging from the solvent/detergent treatment step of an exemplary process of the invention.

In various embodiments, affinity chromatography using a monoclonal antibody specifically binding to is used to further purify a FVIII preparation downstream of filtration, e.g., emerging from the solvent/detergent treatment step of an exemplary process of the invention.

The term “under sterile conditions” as used herein refers to maintaining the sterility of the system, for example by using sterile connecting means of two or more vessels (e.g., bags) from a blood processing set, or refers to a means by which the process does not introduce contamination. For example, as used in the methods described herein, a source unit of blood product such as cryoprecipitate or plasma comprising a tubing for connection to a processing set or container of pathogen inactivation compound comprising a similar tubing may be joined under sterile condition by methods known in the art, for example using a sterile connecting device, which acts to melt or weld the tubing together to provide a sterile flow path between the two containers. In various embodiments, transfer of intermediates and products of the process of the invention between steps is performed under sterile conditions

“International Unit,” or “IU,” is a unit of measurement of the blood coagulation activity (potency) of FVIII as measured by a standard assay such as a one-stage assay. One stage assays are known to the art, such as that described in N Lee, Martin L, et al., An Effect of Predilution on Potency Assays of FVIII Concentrates, Thrombosis Research (Pergamon Press Ltd.) 30, 511 519 (1983). Another standard assay is a chromogenic assay. Chromogenic assays may be purchased commercially, such as the Coatest Factor VIII, available from Chromogeix AB, Molndal, Sweden. In an exemplary embodiment, the activity of FVIII in the final container is from about 20 to about 200 IU/mL.

In an embodiment, the process according to the present invention is performed at a large-scale. In the present context the term “large-scale” relates to the processing of a raw material volume of at least about 1 liter per adsorption cycle, such as at least about 5 liters per adsorption cycle, such as at least about 10 liters per adsorption cycle, such as at least about 25 liters per adsorption cycle, such as at least about 100 liters per adsorption cycle, such as at least about 1000 liters per adsorption cycle and thus distinguish the invention from any analytical and small scale experiments that do not relate to the severe requirements for robustness and reproducibility as in an industrial large-scale production environment. An exemplary method of the invention is a large-scale separation of fibrinogen from FVIII.

A “disease” is a state of health of an animal wherein the animal cannot maintain homeostasis, e.g., haemostasis, and wherein if the disease is not ameliorated then the animal's health continues to deteriorate.

“Haemostasis” is an important physiological process that prevents bleeding following damage (e.g. a rupture) to blood vessels. There are three basic mechanisms that promote haemostasis: (i) vasoconstriction, (ii) platelet aggregation at the rupture site; and (iii) coagulation. During coagulation, damaged endothelial cells release tissue factor (Factor III), which in turn activates Factor VII with the aid of Ca²⁺. Factor XII, which is released by activated platelets, activates Factor XI. Activated Factor VII and Factor XI promote a cascade of enzymatic reactions that lead to the activation of Factor X. Active Factor X (Factor Xa), along with Factor m, Factor V, Ca²⁺, and platelet thromboplastic factor (PF3), activate prothrombin activator. Prothrombin activator converts prothrombin to thrombin, which converts fibrinogen (Factor I) to fibrin, which forms an initial mesh over the site of damage. The initial mesh is then converted to a dense fibrin clot by Factor XIII, sealing the rupture until the site is repaired. During the coagulation cascade, thrombin will also activate Factor VIII, a glycoprotein pro-cofactor that in the circulation is mainly complexed to von Willebrand factor (VWF). Factor VIII interacts with Factor IXa to activate Factor X in the presence of Ca⁺² and phospholipids.

A deficiency in the level of any one or more of the proteins involved in coagulation, including fibrinogen, Factor VIII, and/or von Willebrand factor (VWF) whether congenital or acquired, can lead to insufficient clotting of blood and the risk of hemorrhage. Current treatment options are limited to the administration of a pharmaceutical formulation of one or more therapeutic proteins, with a view to restoring endogenous levels of the protein(s) and maintaining haemostasis.

In various embodiments, the FVIII produced by a method of the invention, or a pharmaceutical formulation thereof, is administered to a subject for prophylaxis to prevent a disease, or to treat a disease in a subject in need of such treatment or prophylaxis. In exemplary embodiments, the disease is a disruption of haemostasis. In various embodiments, the disease is excessive bleeding due to a deficiency in blood clotting. In an exemplary embodiment, the bleed is due to a deficiency in the subject of FVIII.

The term “pharmacologically active” means that a substance so described is determined to have activity that affects a medical parameter or disease state. In an exemplary embodiment, the invention provides a pharmaceutically active FVIII formulation prepared by a method of the invention. An exemplary FVIII formulation of the invention is suitable for infusion. An exemplary pharmacologically active FVIII formulation is pathogen inactivated.

The term “suitable for infusion” refers to any blood product (e.g., FVIII) able to be used for an infusion into a subject (e.g., a human patient) according to medical judgment. In some embodiments, suitability refers to having sufficient biological activity for its intended use, i.e., for use where a transfusion of human coagulation factors is indicated, including, without limitation, control of bleeding associated Factor VIII deficiency, maintenance of hemostasis, treating disseminated intravascular coagulation (DIC) and/or high-volume hemorrhage. In some embodiments, suitability refers to having sufficient safety, e.g., that the product has undergone a treatment that improves product safety (e.g., pathogen inactivation) and/or demonstrates satisfactory performance with respect to one or more safety-related measurements (such as viral or bacterial titer). Photochemical inactivation of pathogens in blood product units using amotosalen and UVA light as described herein is well-established to provide such a blood product (e.g., cryoprecipitate) that is suitable for transfusion into humans. In some embodiments, suitability refers to meeting one or more standards (e.g., having a level of a biological activity or a biological component, a safety criterion, and the like) established by an accrediting agency or regulatory body that governs infusion practices, such as the AABB.

“Pathogen-inactivated” as used herein describes a blood product (e.g., a cryoprecipitate or plasma) that has undergone processing (e.g., by the methods described herein) to inactivate pathogens that may be present. It is understood that a pathogen-inactivated cryoprecipitate or fraction downstream from cryoprecipitate may include a cryoprecipitate or a downstream fraction that has itself undergone pathogen inactivation, or a cryoprecipitate made from a pathogen-inactivated blood product (e.g., plasma, whole blood, and the like). The inactivation of a pathogen may be assayed by measuring the number of infective pathogens (e.g., virus or bacteria) in a certain volume, and the level of inactivation is typically represented by the log reduction in the infectivity of the pathogen, or log reduction in titer. Methods of assaying log reduction in titer, and measurements thereof for pathogen inactivation are known in the art. Methods of assaying log reduction in titer, and measurements thereof for pathogen inactivation are described, for example, in U.S. Pat. No. 7,655,392, the disclosure of which is hereby incorporated by reference as it relates to assays for pathogen inactivation. As such, for any given pathogen, known amounts can be added to a test unit of cryoprecipitate or plasma to assess how much inactivation results from the process, where typically the pathogen inactivation process results in at least about 1 log reduction in titer, or about 2 log, about 3 log, about 4 log, or at least about 5 log reduction in titer. While the methods as described herein are applicable to any pathogen-inactivation treatment, it is desirable that the pathogen-inactivation treatment is capable of inactivating a variety of pathogens to at least 1 log reduction in titer, including a pathogen selected from the group consisting of HIV-1, HBV, HCV, HTLV-1, HTLV-2, West Nile virus, Escherichia coli, Klebsiella pneumoniae, Yersinia enterocolitica, Staphylococcus epidermidis, Staphylococcus aureus, Treponema pallidum, Borrelia burgdorferi, Plasmodium falciparum, Trypanosoma cruzi, and Babesia microti. In various embodiments, the invention provides a method of separating fibrinogen from FVIII and forming a pathogen inactivated FVIII formulation from the product of this method.

As used herein, “pharmaceutically acceptable carrier” includes solvents, dispersion media, coatings, antibacterial and antifungal agents, isotonic and absorption delaying agents, and the like that are physiologically compatible. Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, amino acids (e.g., glycine, proline, etc.), or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the FVIII Compositions comprising such carriers are formulated by well-known conventional methods. Exemplary formulations of the invention include one, two, or more, different amino acids. In an exemplary embodiment, the presence of the amino acid(s) improves the stability of the antibodies, even at high concentrations at which the FVIII is typically not stable in formulations absent the amino acid(s). In various embodiments, the carrier is selected to provide a “stable pharmaceutical formulation”.

As used herein, the term “amino acid” refers to either natural and/or unnatural or synthetic amino acids.

In one embodiment, a stable pharmaceutical formulation contains a FVIII product of a process of the invention and at least one amino acid selected based on the amino acids ability to increase the stability of FVIII and/or reduce solution viscosity. In one embodiment, the amino acid contains a positively charged side chain, such as R, H, and K. In another aspect, the amino acid contains a negatively charged side chain, such as D and E. In another embodiment, the amino acid contains a hydrophobic side chain, such as A, F, I, L, M, V, W, and Y. In another embodiment, the amino acid contains a polar uncharged side chain, such as S, T, N, and Q. In yet another embodiment, the amino acid does not have a side chain, i.e., G.

The term “stable formulation” such as “stable pharmaceutical formulation” as used in connection with the formulations described herein denotes, without limitation, a formulation, which preserves its physical stability/identity/integrity and/or chemical stability/identity/integrity and/or biological activity/identity/integrity during manufacturing, storage and application. Various analytical techniques for evaluating protein stability are available in the art and reviewed in Reubsaet, et al. (1998) J Pharm Biomed Anal 17(6-7): 955-78 and Wang, W. (1999) Int J Pharm 185(2): 129-88. Stability can be evaluated by, for example, without limitation, storage at selected climate conditions for a selected time period, by applying mechanical stress such as shaking at a selected shaking frequency for a selected time period, by irradiation with a selected light intensity for a selected period of time, or by repetitive freezing and thawing at selected temperatures. The stability may be determined by, for example, at least one of the methods selected from the group consisting of visual inspection, SDS-PAGE, IEF, size exclusion liquid chromatography (SEC-HPLC), reversed phase liquid chromatography (RP-HPLC), ion-exchange HPLC, capillary electrophoresis, light scattering, particle counting, turbidity, RFFIT, and kappa/lambda ELISA, without limitation. Exemplary characteristics of use with visual inspection include turbidity and aggregate formation.

In an embodiment, a formulation is considered stable when the FVIII in the formulation (1) retains essentially its entire native physical stability, (2) retains essentially its entire chemical stability and/or (3) retains it biological activity.

Exemplary FVIII formulations of the invention are stable formulations, e.g. stable pharmaceutical formulations.

PIM may be said to “retain its physical stability” in a formulation if, for example, without limitation, it shows no signs of aggregation, precipitation and/or denaturation upon visual examination of color and/or clarity, or as measured by UV light scattering or by size exclusion chromatography (SEC) or electrophoresis, such as with reference to turbidity or aggregate formation. An alternative, cotnpatible definition is a formulation that meeting one or more of the physical stability requirements for an equivalent pharmaceutical product that has achieved marketing approval from one or more regulatory agency (e.g,, FDA, EMA, etc.).

FVIII may be said to “retain its chemical stability” in a formulation, if, for example, without limitation, the chemical stability at a given time is such that there is no significant modification of the FVIII by bond formation or cleavage resulting in a new chemical entity. In a further embodiment, chemical stability can be assessed by detecting and quantifying chemically altered forms of the FVIII. Chemical alteration may involve, example, without limitation, size modification (e.g. clipping) which can be evaluated using size exclusion chromatography, SDS-PAGE and/or matrix-assisted laser desorption ionization/time-of-flight mass spectrometry (MALDI/TOF MS). Other types of chemical alteration include, for example, without limitation, charge alteration (e.g. occurring as a result of deamidation), which can be evaluated by ion-exchange chromatography, for example. Oxidation is another commonly seen chemical modification. An alternative, compatible definition is a FVIII formulation meeting one or more of the stability requirements for an equivalent pharmaceutical product that has achieved marketing approval from one or more regulatory agency (e.g., FDA, EMA, etc.).

In an embodiment, MIT may be said to “retain its biological activity” relative to native FVIII in a pharmaceutical formulation, if, for example, without limitation, the biological activity of FVIII, at a given time is between about 50% and about 200%, or alternatively between about 60% and about 170%, or alternatively between about 70% and about 150%, or alternatively between about 80% and about 125%, or alternatively between about 90% and about 110%, of the biological activity exhibited at the time the formulation was prepared as determine. In a further embodiment, FVIII may be said to “retain its biological activity” in a pharmaceutical formulation, if, for example, without limitation, the biological activity of FVIII prepared by a method of the invention, at a given time is at least about 50%, 55%, 60%, 65%, 70%, 75%, 80%, 85%, 90%, 95% or at least about 100% of the activity of a reference standard in an art-recognized FVIII activity assay. An alternative, compatible definition is a FVIII formulation meeting one or more of the biologic activity requirements for an equivalent pharmaceutical product that has achieved marketing approval from one or more regulatory agency (e.g., FDA, EMA, etc.).

A “therapeutically effective amount” of FVIII prepared by a method of the invention refers to an amount effective, at dosages, dosing intervals, and for periods of time necessary, to achieve the desired therapeutic result. A therapeutically effective amount of FVIII may vary according to factors such as the disease state, age, sex, and weight of the individual, and the ability of FVIII to elicit a desired response in the individual. In exemplary embodiments, treating a subject infected with a diseased related to haemostasis with a therapeutically effective amount of a pharmaceutical formulation comprising FVIII prepared by a method of the invention of the invention reduces the risk of mortality. In some embodiments, treatment of an infected subject with a therapeutically effective amount of pharmaceutical formulation of the invention speeds time to recovery for the subject.

In an exemplary embodiment, the invention provides a unit dosage formulation of FVIII prepared by a method of the invention. The unit dosage contains a therapeutically effective amount of the FVIII.

The terms “dose” and “dosage” are used interchangeably herein. A dose refers to the amount of active ingredient given to an individual at each administration. The dose will vary depending on a number of factors, including frequency of administration; size and tolerance of the individual; severity of the condition; risk of side effects; and the route of administration. One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for infusion.

As used herein, “administering” means, intravenous, intraperitoneal, intramuscular, intralesional, or subcutaneous administration, intrathecal administration, or instillation into a surgically created pouch or surgically placed catheter or device to the subject.

As used herein, the term “subject” includes human subjects.

As used herein, the term “therapy,” “treatment,” and “amelioration” refer to any reduction in the severity of symptoms arising from a condition associated with the absence of, lack of function or dysfunction of a blood protein. As used herein, the terms “treat” and “prevent” are not intended to be absolute terms. Treatment can refer to any delay in onset, amelioration of symptoms, improvement in patient survival, increase in survival time or rate, e.g., (i) slowing, stopping or reversing the progression of one or more of the symptoms, (ii) slowing, stopping or reversing the progression of illness underlying such symptoms, (iii) reducing or eliminating the likelihood of the symptom's recurrence, and/or (iv) slowing the progression of, lowering or eliminating the disruption in haemostasis. The effect of treatment can be compared to an individual or pool of individuals not receiving the treatment.

As used herein, the term “prevent” refers to a decreased likelihood or reduced frequency of symptoms arising from a condition associated with the lack of function or dysfunction of a blood protein.

As used herein, an “isolated” FVIII is human FVIII that is at least about 90% pure (i.e., does not contain more than 10% protein impurity). Preferably, isolated human FVIII is at least about 95%, 98%, 99% or at least about 99.5% pure.

In an exemplary embodiment, the FVIII produced by the method of the invention is at least as pure as that obtained in accepted industtial methods for preparing this protein for administration to humans.

In various embodiments, the invention provides isolated FVIII, which is isolated by a method of the invention.

As used herein, the term “about” denotes an approximate range of plus or minus 10% from a specified value. For instance, the language “about 20%” encompasses a range of 18-22%. As used herein, about also includes the exact amount. Hence “about 20%” means “about 20%” and also “20%.” As used herein, “about” refers to a range of at or plus or minus up to about 10% of the specified value.

Throughout this specification and claims, the word “comprise,” or “variations such as comprises” or “comprising,” will be understood to imply the inclusion of a stated integer or group of integers but not the exclusion of any other integer or group of integers.

### III. Embodiments

Reference will now be made in detail to implementation of exemplary embodiments of the present disclosure. Those of ordinary skill in the art will understand that the following detailed description is illustrative only and it is not intended to be in any way limiting. Other embodiments of the present disclosure will readily suggest themselves to such skilled persons having benefit of this disclosure.

In the interest of clarity, not all of the routine features of the implementations described herein are shown and described. It will be appreciated that, in the development of any such actual implementation, numerous implementation-specific decisions are made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Many modifications and variations of the exemplary embodiments set forth in this disclosure are made without departing from the spirit and scope of the exemplary embodiments, as will be apparent to those skilled in the art. The specific exemplary embodiments described herein are offered by way of example only, and the disclosure is to be limited only by the terms of the appended claims, along with the full scope of equivalents to which such claims are entitled.

### A. Methods

In an exemplary embodiment, the invention provides a method of separating plasma cryoprecipitate comprising a blood coagulation factor and fibrinogen into a first fraction comprising the blood coagulation factor and a second fraction containing the fibrinogen, the method comprising: (a) contacting the plasma cryoprecipitate with solid SiO₂, thereby adsorbing the fibrinogen onto the solid SiO₂; and (b) separating the fibrinogen adsorbed onto the solid from the blood factor, thereby forming the first fraction and the second fraction.

An exemplary embodiment further comprises (c), prior to (a), suspending the cryoprecipitate in water, forming a cryoprecipitate suspension. The suspension is an exemplary “starting composition” for the method described herein.

The cryoprecipate, or other source of FVIII, and water are combined in any useful ratio, for example, in a cryoprecipitate:water ratio of from about 1:2 to about 1:7, e.g., about 1.3 to about 1:6, in the cryoprecipitate suspension, In various embodiments, the cryoprecipitate:water ratio is from about 1:3.5 to about 1:5 in the cryoprecipitate suspension.

In various embodiments, it is advantageous to include a salt in the cryoprecipitate suspension. An exemplary salt is a divalent metal ion salt. In an exemplary embodiment, the salt includes a divalent cation, e.g., CaCl₂. The salt is present in any useful amount. In an exemplary embodiment in which the divalent metal salt is CaCl₂, the CaCl₂ is present in from about 40 μM to about 70 mM, e.g., from about 100 μM to about 60 mM, e.g., from 250 μM to about 50 mM in the cryoprecipitate suspension.

The silica mixed with the cryoprecipitate suspension can be any silica useful for the purpose of removing fibrinogen from the cryoprecipitate suspension, decreasing the concentration of this protein in the cryoprecipitate suspension. In various embodiments, the SiO₂ is fumed SiO₂, e.g., fumed SiO₂ is hydrophilic colloidal SiO₂. In an exemplary embodiment, the SiO₂ is an Aerosil product, e.g., Aerosil® 380 or an analogous adsorptive material.

In certain embodiments of the methods provided herein, the amount of fibrinogen in the FVIII preparation is reduced by at least about 10% relative to the amount of fibrinogen in the starting plasma or Cryoprecipitate. In another embodiment, the amount of fibrinogen is reduced by at least about 25% from the starting plasma or Cryoprecipitate. In another embodiment, the amount of fibrinogen is reduced by at least about 50%, or at least about 60% from the amount present in the starting plasma or Cryoprecipitate. In another embodiment, the amount of fibrinogen is reduced by at least 75%. In another embodiment, the amount of fibrinogen is reduced by at least 90%. In yet other embodiments, the amount of fibrinogen is reduced by at least 5%, or by at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or to levels below the detection limit of the test system.

In an exemplary embodiment, following treatment with silicon dioxide, the intermediate composition has an amount of fibrinogen reduced by about 60% compared to the starting plasma or starting Cryoprecipitate.

In an exemplary embodiment, the final composition at the end of the purification cycle has an amount of fibrinogen reduced by at least about 99%, or below the limits of detection, relative to the fibrinogen content of the starting plasma or Cryoprecipitate.

The solid SiO₂ is mixed with the cryoprecipitate in any useful ratio and amount. Generally, the amount of finely divided silicon dioxide (SiO₂) required for the methods described herein will vary dependent on several factors, including without limitation, the total amount of protein present in the starting composition, the concentration of FVIII in the composition, the amount of fibrinogen in the starting composition, and the solution conditions (e.g., pH, conductivity, etc.). For example, SiO₂ may be added to a starting composition at a concentration between about 0.01 g/g protein and about 10 g/g protein. In another embodiment, SiO₂ may be added to a starting composition at a concentration between about 1 g/g protein and about 5 g/g protein. In another embodiment, SiO₂ may be added to a starting composition at a concentration between about 2 g/g protein and about 4 g/g, protein. In one embodiment, SiO₂ is added at a final concentration of at least about 1 g per gram total protein. In an embodiment, fumed silica is added at a concentration of at least about 2 g per gram total protein. in a specific embodiment, fumed silica is added at a concentration of at least about 2.5 g per gram total protein. In various embodiments, SiO₂ may be added to a target composition at a concentration between about 0.01 g/g protein and about 5 g/g protein. In an exemplary embodiment, SiO₂ may be added to a target composition at a concentration between about 0.02 g/g protein and about 4 g/g protein. In one embodiment, SiO₂ is added at a final concentration of at least 0.1 g per gram total protein. In another specific embodiment, fumed silica is added at a concentration of at least 0.2 g per gram total protein. In another specific embodiment, fumed silica is added at a concentration of at least 0.25 g per gram total protein. In yet other specific embodiments, finely divided silicon dioxide is added at a concentration of at least about 0.01 g/g total protein or at least 0.02 g, 0.03 g, 0.04 g, 0.05 g, 0.06 g, 0.07 g, 0.08 g, 0.09 g, 0.1 g, 0.2 g, 0.3 g, 0.4 g, 0.5 g, 0.6 g, 0.7 g, 0.8 g, 0.9 g, 1.0 g, 1.5 g, 2.0 g, 2,5 g, 3.0 g, 3.5 g, 4,0 g, 4.5 g, 5.0 g, 5.5 g, 6.0 g, 6.5 g, 7.0 g, 7.5 g, 8,0 g, 8.5 g, 9.0 g, 9.5 g, or at least about 10.0 g, or more gig total protein.

In various embodiments, the SiO₂ is present in the suspension in from about 5 g to about 30 g of SiO₂ per kilogram of the cryoprecipitate suspension. In an exemplary embodiment, the SiO₂ is present in the suspension in from about 10 g to about 20 g of SiO₂ per kilogram of the suspension.

In certain embodiments of the invention a further solid material, e.g., a filter aid, is combined with the cryoprecipitate suspension.

In certain embodiments a filter aid, for example Celpure C300 (Celpuret or Hyflo-Super-Cel (World Minerals), to facilitate filtration. Filter aid can be added at a final concentration of from about 0.01 kg/kg starting composition to about 1.0 kg/kg starting composition, or from about 0.02 kg/kg starting composition to about 0.8 kg/kg starting composition, or from about 0.03 kg/kg starting composition to about 0.7 kg/kg starting composition. In other embodiments, filter aid can be added at a final concentration of from about 0.01 kg/kg starting composition to about 0.07 kg/kg starting composition, or from about 0.02 kg/kg starting composition to about 0.06 kg/kg starting composition, or from about 0.03 kg/kg precipitate to about 0.05 kg/kg starting composition. In certain embodiments, the filter aid will be added at a final concentration of about 0.01 kg/kg starting composition, or about 0.02, 0.03, 0.04, 0.05, 0.06, 0.07, 0.08, 0.09. 0.1, 0.2, 0.3, 0.4, 0.5, 0.6, 0,7, 0,8, 0,9, or 1.0 kg/kg starting composition.

In an exemplary embodiment, the filter aid is present in the cryoprecipitate suspension in from about 2 g to about 10 g of filter aid per kilogram of cryoprecipitate suspension. In various embodiments the filter aid is present in the cryoprecipitate suspension in from 4 g to about 8 g per kilogram of the cryoprecipitate suspension, In an exemplary embodiment, the filter aid is present in the cryoprecipitate suspension in from about 5 g to about 6.5 g per kilogram of the cryoprecipitate suspension, e.g., from about 5.2 g to about 6 g/kg, e,g., from about 5.4 g to about 5.8 g/kg of the cryoprecipitate suspension.

In some embodiments, the filter aid is added after the silica dioxide treatment, before silicon dioxide treatment or concurrent with silicon dioxide treatment to facilitate subsequent filtration.

In general, the various suspensions formed during the process, e.g., the cryoprecipitate suspension, are stirred to homogeneity in the course of the process.

The solid SiO₂ and the starting composition, e.g., the cryoprecipitate suspension (or Fraction II-III suspension) are mixed under conditions allowing fibrinogen to adsorb onto the SiO₂. In various embodiments, the starting composition/SiO₂ suspension is at a temperature of from about 15° C. to about 37° C., e.g., from about 20° C. to about 32° C.

Following the step of mixing the starting composition with one or both of silica and a, filter aid, the method further comprises (d) passing the cryoprecipitate suspension through a filtration device, thereby forming a filter cake and a filtrate.

In general, any filtration device is of use which is suitable for separating a essentially all of the silica adsorbed fibrinogen from the mixture of the cryoprecipitate suspension. In an exemplary embodiment, the filtration device is a mesh screen, An exemplary mesh screen has pores of from about 100 μm to about 400 μm in diameter. In an exemplary embodiment, the mesh screen has pores of greater than about 100 μm.

The filtration methodology is optionally tangential, dead end or any other useful filtration methodology. It is within the purview of those of skill in the art to devise a filtration device and method appropriate to accomplish the goals of the filtration step of the invention.

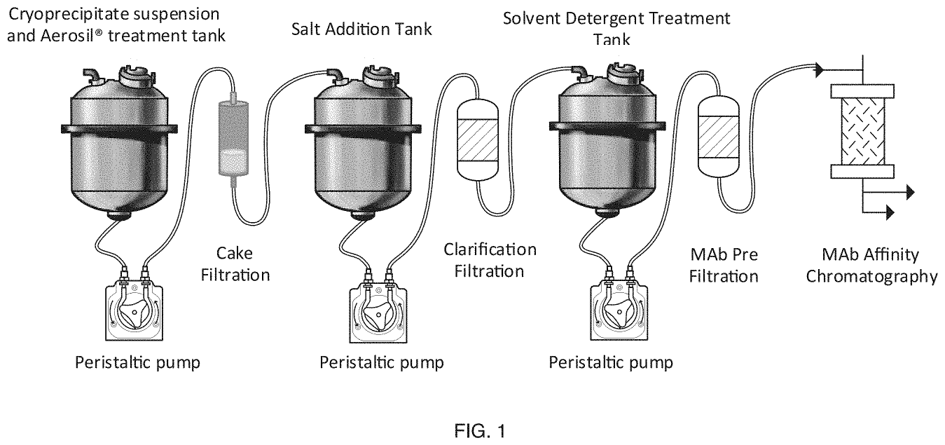

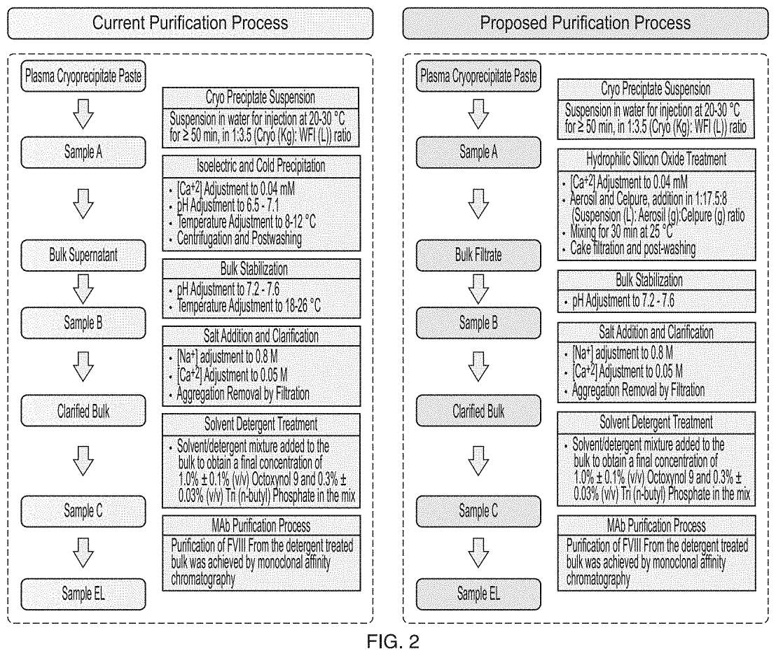

During filtration, FVIII not adsorbed onto or otherwise associated with the silica is collected in a first filtrate. Following filtration, the filter cake is optionally washed with a liquid, e.g, an aqueous solution of one or more salt. In various embodiments, collecting this wash recovers a fraction of FVIII from the filter cake. In an exemplary embodiment, the amount of FVIII recovered in this fraction is essentially quantitative with respect to the amount of FVIII entrained in the filter cake. In an embodiment, the FVIII containing wash is combined with the FVIII in solution (not adsorbed onto or otherwise associated with the silica) to form the Bulk Filtrate (FIG. 1, FIG. 2). An exemplary liquid is an aqueous liquid containing at least one metal ion salt, e.g., a singly charged metal ion. In an exemplary embodiment, the metal ion salt is NaCl.

In an exemplary embodiment, the liquid is an aqueous salt solution, e.g., aqueous NaCl. In an exemplary embodiment, NaCl is present at from about 0.1M to about 0.2M, e.g., from about 0.13M to about 0.16M, e.g, from about 0.14M to about 0.15M. In an exemplary embodiment, the NaCl concentration is about 0.145M. The inventors have discovered that the ionic content of the wash liquid is relevant to product recovery if the ionic concentration is too high, impurities are leached from the silicon dioxide, and if the ionic concentration is too low, recoverable FVIII remains adsorbed on the silicon dioxide.

In various embodiments, the invention does not use centrifugation for separating the fibrinogen adsorbed onto the solid SiO₂ from the blood factor, forming the first fraction and the second fraction. In certain embodiments, it may be advantageous to utilize centrifugation to improve separation of fibrinogen adsorbed onto the solid SiO₂ from the blood factor, forming the first fraction and the second fraction.

Under certain conditions the Bulk Filtrate or one of its components, e.g., first filtrate, includes particulate matter, e.g., aggregations. This particulate matter is reduced or eliminated by filtration of the Bulk Precipitate. An exemplary filtration involves, (e) filtering the first filtrate through a 0.2 μm filter, forming a collected FVIII fraction and a filter cake adsorbing fibrinogen.

The character of the Bulk Filtrate, or the filtrate product of step (e) can be adjusted by the addition of salts or other additives. In an exemplary embodiment, a metal ion salt is added. In one embodiment, the metal ion salt is a salt of a singly charged metal ion, e.g., Na,⁺ e.g., NaCl.

An additive to the Bulk Filtrate or the filtrate product of step (e) is, in exemplary embodiments, present in an amount from about 100 mM to about 200 mM. In an exemplary embodiment, the additive is a salt of a metal ion in this amount, e.g., NaCl. For purposes of exemplification the amount of the additive is about 150 mM.

In an exemplary embodiment, prior to step (e), the method includes (g), prior to (e), adding calcium chloride to the first filtrate to a final concentration of from about 0.045 M to about 0.055 M. In an exemplary embodiment, the calcium chloride is added to the first filtrate to a final concentration of about 0.050 M.

In an exemplary embodiment, the CaCl₂ is added from a stock CaCl₂ solution, e.g., a 5M CaCl₂ solution.

In an exemplary embodiment, the Clarified Bulk solution is submitted to a homogeneous solvent/detergent mixture for viral reduction. An exemplary solvent/detergent mixture is octoxynol and tri(n-butyl)phosphate. In an exemplary embodiment, the concentration of the solvent/detergent is one that is required by a national agency governing marketing approval of pharmaceuticals (e.g., FDA), An exemplary solvent/detergent mixture includes 1.0% ±0.1% (v/v) octoxynol and 0.3% ±0.03% (v/v) tri(n-butyl)phosphate.

In various embodiments, the method further comprises the viral reduction mixture above through an aggregation removal filter, forming a second filtrate.

In an exemplary embodiment, the method comprises (k), loading the second filtrate onto an affinity chromatography packing in a chromatography column pre-equilibrated with an equilibration buffer, said affinity chromatography packing comprising a solid support with a monoclonal antibody specifically binding the blood coagulation factor, immobilizing the blood coagulation factor on the affinity chromatography packing. This step is followed by (l), washing the affinity chromatography packing with a wash buffer, removing materials not bound or weakly bound to the affinity chromatography packing. To collect the purified blood factor, the method includes (m), eluting the blood coagulation factor from the affinity chromatography packing with an elution buffer, and collecting at least one fraction of the elution buffer containing the blood coagulation factor. See, e.g., U.S. Pat. No. 5,470,954.

In various embodiments, the method of the invention is a large-scale enrichment of a blood factor and separation of the blood factor from fibrinogen.

In various embodiments, the invention further includes a step for recovering fibrinogen from the filter cake. An exemplary method includes eluting the fibrinogen off the filter cake with an appropriate eluent and collecting the fibrinogen so eluted.

In exemplary embodiment, according to those described herein, the blood coagulation factor is FVIII.

FIG. 1 shows an exemplary apparatus for carrying out an exemplary process of the invention. The cryoprecipitate suspension is contained in a SiO_{2,} e.g., Aerosil, treatment tank, which is in fluidic communication with a salt addition tank. After treatment of the cryoprecipitate suspension with silica, the resulting suspension is transferred via a filtering means to a. second tank, a salt addition tank. In the salt addition tank the sodium concentration is adjusted to about 0.8 M, and the calcium concentration to about 0.05 M. The resulting suspension is optionally transferred from the salt addition tank via a clarifying filtering means to a solvent/detergent tank where it is submitted to a viral reduction step. An exemplary viral reduction step includes forming a solution with tri-(n-butyl) phosphate (e.g., about 1%) and Octoxynol 9 (e.g., about 0.3%). The process may he completed here or may be continued in an apparatus having an optional pre-filtration device to remove particulates prior to loading the solution from the solvent/detergent treatment tank onto an affinity column comprising immobilizing MAbs binding specifically to FVIII. As will be appreciated by those of skill in the art, the solutions and suspensions generated in the apparatus during the process are conveniently transferred from stage to stage by means of one or more pump, e.g., peristaltic pump.

An exemplary process scheme for a method of the invention is set out in FIG. 2. An exemplary known process is set forth in FIG. 2, Plasma cryoprecipitate paste is suspended in water for injection in a ratio of 1 (kg):3.5(L) cryoprecipitate paste: water, forming Sample A. The Ca²⁺ concentration of Sample A is adjusted to 0.04 M with CaCl₂, and silica and a filter aid are added to the mixture, which is stirred for about 30 min at 25° C., and filtered, producing Bulk Filtrate, the pH of which is adjusted to 7.2-7.6, forming Sample B. Sample B is submitted to salt addition and clarification, using NaCl, adjusting the Na⁺ content of Sample B to 0.8 M, and CaCl₂, adjusting the Ca²⁺ concentration of Sample B to 0.05 M; any aggregate is removed by filtration, resulting in Clarified Bulk. This solution is submitted to a viral reduction step using solvent/detergent treatment. A solvent (tri-(n-butyl) phosphate) and detergent (Octoxynol 9) were added to Clarified Bulk to a final concentration of 1.0% (v/v) and 0.3% (v/v), respectively. forming Sample C. The process may be completed here or, in some embodiments, Sample C is loaded onto a MAb column specifically binding FVIII, and purified by affinity chromatography.

### B. Compositions

In an exemplary embodiment, the invention provides a blood coagulation factor, e.g., FVIII preparation prepared according to the method set forth herein. An exemplary blood coagulation factor preparation is formulated as a pharmaceutical formulation in which the factor is combined with a pharmaceutically acceptable carrier. An exemplary preparation and/or formulation is pathogen inactivated, e.g.. by solvent/detergent treatment. The pharmaceutical formulations are generally suitable for infusion into a subject in need of such infusion. Exemplary formulations are formatted as a unit dosage formulation and include a therapeutically effective amount of the factor. The invention also provides stable pharmaceutical formulations of the factor produced by a method of the invention.

In an exemplary embodiment, the preparation and/or formulation are characterized by one or more parameters that are essentially the same as those of preparations and/or formulations of the factor produced by methods other than the method set forth herein. In an exemplary embodiment, the preparation and/or formulation is a pharmaceutical product or precursor to a product that has achieved marketing approval from one or more regulatory agency (e.g., FDA, EMA, etc.).

An exemplary factor is FVIII. U.S. Pat. No. 5,763,401 (EP 818 204) describes a therapeutic FVIII formulation without albumin, comprising 15-60 mM sucrose, up to 50 mM NaCl, up to 5 mM calcium chloride, 65-400 mM glycine, and up to 50 mM histidine.

U.S. Pat. No. 5,733,873 (EP 627 924) to Osterberg (assigned to Pharmacia & Upjohn) discloses formulations which include between 0.01-1 mg/ml of a surfactant. Other formulations have also been described. U.S. Pat. No, 4,877,608 (EP 315 968), U.S. Pat. No. 5,605,884 (EP 0 314 095) teaches the use of formulations with relatively high concentrations of sodium chloride, WO 2010/054238, EP 1 712 223, WO 2000/48635, WO 96/30041, WO 96/22107, WO 2011/027152, EP 2 361 613, EP 0 410 207, EP 0 511 234, U.S. Pat. No. 5,565,427, EP 0 638 091, EP 0 871 476, EP 0 819 010, U.S. Pat. No. 5,874,408, US 2005/0256038, US 2008/0064856, WO 2005/058283, WO 2012/037530, WO 2014/026954 and US 2017/0252412. A FVIII preparation of the invention can be incorporated into the formulations disclosed in these or other references.

### C. Methods f Treating

In various embodiments, the invention provides a method of treating a disease associated with a dysfunction in haemostasis. An exemplary treatment is administered during an uncontrolled bleeding event. The invention is described by way of example, through reference to FVIII but it is not so limited.

In an exemplary embodiment, the subject needed treatment (or prophylaxis) is administered a dose of FVIII, produced by a method of the present disclosure, of approximately 30 IU/kg to 50 IU/kg.

The dosage regimen involved in a method for treating a condition described herein will be determined by the attending physician, considering various factors which modify the action of drugs, e.g. the age, condition, body weight, sex and diet of the patient, the severity of any infection, time of administration and other clinical factors. In one aspect, formulations of the disclosure are administered by an initial bolus followed by a continuous infusion to maintain therapeutic circulating levels of drug product. As another example, the inventive compound is administered as a one-time dose. Those of ordinary skill in the art will readily optimize effective dosages and administration regimens as determined by good medical practice and the clinical condition of the individual patient. The frequency of dosing depends on the pharmacokinetic parameters of the agents and the route of administration. The optimal pharmaceutical formulation is determined by one skilled in the art depending upon the route of administration and desired dosage. See for example, REMINGTON'S PHARMACEUTICAL SCIENCES, 18th Ed. (1990, Mack Publishing Co., Easton, Pa. 18042) pages 1435-1712, the disclosure of which is hereby incorporated by reference. Such formulations influence the physical state, stability, rate of in vivo release, and rate of in vivo clearance of the administered agents. Depending on the route of administration, a suitable dose is calculated according to body weight, body surface area or organ size. Appropriate dosages may be ascertained through use of established assays for determining blood level dosages in conjunction with appropriate dose-response data. The final dosage regimen is determined by the attending physician, considering various factors which modify the action of drugs, e.g. the drug's specific activity, the severity of the damage and the responsiveness of the patient, the age, condition, body weight, sex and diet of the patient, the severity of any infection, time of administration and other clinical factors. As studies are conducted, further information will emerge regarding the appropriate dosage levels and duration of treatment for various diseases and conditions.

The following Examples are offered to illustrate exemplary embodiments of the invention and do not define or limit its scope,

### EXAMPLES

### Example 1

Frozen cryoprecipitate was suspended in water at 20 to 32° C. in the ratio of 1:3.5 to 1:5 of cryoprecipitate to water. Calcium Chloride was added to a concentration of 40 μm. An amount of 10 to 20 g of hydrophilic colloidal silicon oxide (such as Aerosil® 380) per Kg of suspension and 4 to 8 g of filter aid (such as Celpuret C300) per Kg of suspension were added and mixed until fully homogenized. The homogenized suspension was filtered using mesh screen, 100-400 μm pore size. .At the end of filtration, the cake was post-washed with saline solution to maximize recovery. Sodium chloride and calcium chloride were added to the filtrate to obtain final concentrations of 0.8 M and 0.05 M, respectively and the solution was then filtered through a clarifying 0.2 μm filter.

Further purification of the product of the process above was effected as follows. A homogenous solvent/detergent mixture was prepared and slowly added to the solution, while mixing. The to obtain a final concentration of 1.0% ±0.1% (v/v) Octoxynol 9 and 0.3% ±0.03% (v/v) Tri (n-butyl) Phosphate in the mix. Afterward the suspension was filtered through an aggregation removal filter prior to loading into a MAb column that has been equilibrated with MAb Equilibration Buffer. Afterward the MAb column was washed by the MAb Wash Buffer and Factor VIII was then eluted from the column with MAb Elution Buffer.

### Example 2

Frozen cryoprecipitate obtained from human plasma was suspended in Mili-Q water at 24±1° C. in the ratio of 1:3.5 of cryoprecipitate (Kg): Mili-Q water (L)). Sufficient calcium chloride was added to obtain a minimum calcium concentration of 40 ᵢnn. An amount of 17.5 g of Hydrophilic colloidal silicon oxide, Aerosil® 380, and 8 g of filter aid, Celpure® C300, were added to each Kg of the suspension and mixed for 30 min at 400 rpm at room temperature.

The homogenized suspension was filtered using stainless-steel mesh screen, 150-250 μm pore size, as a support, and then the formed cake was washed with saline solution. Sodium chloride and calcium chloride were added to the filtrate to obtain final concentrations of 0.8 M and 0.05 M, respectively and the solution was then filtered through a clarifying 0.2 μm filter.

A homogenous solvent/detergent mixture of Octoxynol 9 and Tri (n-butyl) phosphate was prepared and slowly added to the solution, while mixing. The amount of solvent/detergent mixture added to the protein solution is calculated to obtain a final concentration of 1.0% ±0.1% (v/v) Octoxynol 9 and 0.3% ±0.03% (v/v) Tri (n-butyl) Phosphate in the mix. The Cryo-detergent solution is thoroughly mixed for 60 minutes to assure homogeneity and viral inactivation. Afterward the suspension was filtered through an aggregation removal filter prior to loading onto a MAb column that has been equilibrated with MAb Equilibration Buffer. Afterward the MAb column was washed with a minimum of 40 column volumes of MAb Wash Buffer and Factor VIII. was then eluted from the column with MAb Elution Buffer.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. The present invention has been illustrated by reference to various exemplary embodiments and examples. As will be apparent to those of skill in the art other embodiments and variations of this invention may be devised by others skilled in the art without departing from the true spirit and scope of the invention. The appended claims are to be construed to include all such embodiments and equivalent variations.

The disclosures of each and every patent, patent application, and publication cited herein are hereby incorporated herein by reference in their entirety.

What is claimed is:

1. A method of separating plasma cryoprecipitate comprising a blood coagulation factor and fibrinogen into a first fraction comprising a blood coagulation factor isolate and a second fraction containing the fibrinogen, the method comprising:
(a) contacting the plasma cryoprecipitate with solid Si02, thereby adsorbing the fibrinogen onto the solid Si02; and
(b) separating the fibrinogen adsorbed onto the solid Sith from the blood factor, thereby forming the first fraction and the second fraction.

2. The method according to claim 1, wherein the SiO₂ is fumed SiO₂.

3. The method according to claim 2, wherein the fumed SiO₂ is hydrophilic colloidal SiO₂.

4. The method according to claim 1, further comprising (c), prior to (a), suspending the cryoprecipitate in water, forming a cryoprecipitate suspension.

5. The method according to claim 4, wherein the water is from about 15° C. to about 37° C.

6. The method according to claim 5, wherein the water is from about 20° C. to about 32° C.

7. The method according to claim 4, wherein the cryoprecipitate and water are in a ratio of from about 1:2 to about 1:7 in the cryoprecipitate suspension.

8. The method according to claim 7, wherein the cryoprecipitate and water are in a ratio or from about 1:3.5 to about 1:5 in the cryoprecipitate suspension.

9. The method according to claim 4, wherein the cryoprecipitate suspension further comprises CaCl₂.

10. The method according to claim 9, wherein the CaCl₂ is present in from about 40 μM to about 50 mM in the cryoprecipitate suspension.

11. The method according to claim 10, wherein the CaCl₂ is present in from about 0.045 M to about 0.055 M in the cryoprecipitate suspension.

12. The method according to claim 4, wherein the SiO₂ is present in the suspension in from about 5 g to about 30 g of SiO₂ per kilogram of the suspension.

13. The method according to claim 12, wherein the SiO₂ is present in the suspension in from about 10 g to about 20 g of SiO₂ per kilogram of the suspension.

14. The method according to claim 4, wherein the cryoprecipitate suspension further comprises from about 2 g to about 10 g of filter aid per kilogram of cryoprecipitate suspension.

15. The method according to claim 14, wherein the filter aid is present in the cryoprecipitate suspension in from about 4 g to about 8 g per kilogram of the cryoprecipitate suspension.

16. The method according to claim 4, wherein the cryoprecipitate suspension is mixed to homogeneity.

17. The method according to claim 16, further comprising (d) passing the cryoprecipitate suspension through a filtration device, thereby forming a filter cake and a filtrate.

18. The method according to claim 17, wherein the filtration device is a mesh screen.

19. The method according to claim 18, wherein the mesh screen has pores of from about 100 μm to about 400 μm in diameter.

20. The method according to claim 17, further comprising (e) following (d), washing the filter cake with an aqueous wash solution.

21. The method according to claim 18, wherein the aqueous wash solution is sodium chloride solution.

22. The method according to claim 21, further comprising (e) filtering the filtrate through a 0.2 μm filter, forming a first filtrate.

23. The method according to claim 20, further comprising (f), prior to (e), adding sodium chloride to the first filtrate.

24. The method according to claim 23 wherein the sodium chloride is added to the first filtrate to a final concentration of from about 100 mM and about 200 mM

25. The method according to claim 22, wherein the sodium chloride is added to the first filtrate to a final concentration of about 150 mM.

26. The method according to claim 22, further comprising (g), prior to (e), adding calcium chloride to the first filtrate to a final concentration of from about 0.45 M to about 0.55 M.

27. The method according to claim 23, wherein the calcium chloride is added to the first filtrate to a final concentration of about 0.050 M.

28. The method according to claim 23, further comprising (h), prior to (e), adding calcium chloride to the first filtrate.

29. The method according to claim 1, further comprising (i) contacting the first filtrate of (e) with a homogeneous solvent/detergent mixture, forming a first filtrate suspension.

30. The method according to claim 29, wherein the homogeneous solvent/detergent mixture is octoxynol and tri(n-butyl)phosphate.

31. The method according to claim 30, wherein the homogeneous solvent/detergent mixture is 1.0% ±0.1% (v/v) octoxynol and 0.3% ±0.03% (v/v) tri(n-butyl)phosphate.

32. The method according to claim 29, further comprising (j) filtering the first filtrate suspension through an aggregation removal filter, forming a second filtrate.

33. The method according to claim 1, wherein the blood coagulation is Factor VIII.

34. The method according to claim 1, wherein centrifugation is not used in separating the fibrinogen adsorbed onto the solid SiO₂ or Al(OH)₃ from the blood factor, forming the first fraction and the second fraction.

35. The method according to claim 1, wherein centrifugation is used in separating the fibrinogen adsorbed onto the solid SiO₂ from the blood factor, forming the first fraction and the second fraction.

36. A blood coagulation factor isolate prepared by the method according to claim 1.

37. The blood coagulation factor isolate prepared by the method according to claim 1, wherein said blood coagulation factor isolate contains less fibrinogen than the plasma cryoprecipitate.

33. The blood coagulation factor isolate prepared by the method according to claim 1, wherein the blood coagulation factor is FVIII.
